(19) United States
(12) Patent Application Publication
(10) Pub. No.: US 2003/0157194 A1
(43) Pub. Date: Aug. 21, 2003

(54) Method for treating cellulite

(76) Inventor: Ralph L. Bass, Chapel Hill, NC (US)

Correspondence Address: Jennifer L. Skord, 133 Country Lane, Pittsboro, NC, 27312, US

(21) Appl. No.: 10/078,614

(22) Filed: Feb. 19, 2002

(51) Int. Cl. A61K 33/14

(52) U.S. Cl. 424/679; 424/680

(57) ABSTRACT

A method for the topical treatment of skin affected with cellulite. Sodium chloride, potassium chloride, or a combination thereof, in a solid block formulation, is topically applied to the affected skin by gently gliding the solid block over the affected skin in a manner insufficient to cause abrasion and debridement of the affected skin. Preferably employed is a solid block of about 95 to about 100% by weight sodium chloride. Also preferably, the block of sodium chloride and/or potassium chloride is free of a carrier and/or other topical cellulite-treatment medicaments and/or cosmetics. The applied sodium chloride and/or potassium chloride is left on the skin.

### TECHNICAL FIELD

The present invention relates, in general, to a method for the topical treatment of cellulite. More particularly, the present invention relates to an improved method for the topical treatment of human skin affected with cellulite by gently gliding over the affected skin sodium chloride (hereafter referred to by its chemical name, NaCl), potassium chloride (hereafter referred to by its chemical name, KCl), or a combination thereof.

### BACKGROUND OF THE INVENTION

Cellulite is a skin condition known as dimpling of the skin, typically the skin of the thighs and buttocks. This condition is known to affect women more frequently than men. See, Rosenbaum et al., “An Exploratory Investigation of the Morphology and Biochemistry of Cellulite”, Vol. 1, No. 7, Plastic and Reconstructive Surgery, pp. 1934-1939 (June, 1998). Cellulite can also occur in other skin areas, such as the triceps area of the upper arm, and/or the area along the body sides between each armpit and the waist.

Two common terms for cellulite are “mattress phenomenon” or “orange-peel phenomenon”. See, Scherwitz et al., “So-Called Cellulite”, Vol. 4, No. 3, Journal of Dermatological Surgery and Oncology, pp. 230-243 (March, 1978). Cellulite is also referred to as the cottage cheese phenomenon. The pinch test, namely use of the fingers to pinch the skin and thus to create a fold of the skin in the thighs and/or buttocks area (and/or other skin area that is a cellulite-affected area), is typically employed for determining that cellulite is present. See, Numberger et al., “So-Called Cellulite: An Invented Disease”, Vol. 4, No. 3, Journal of Dermatological Surgery and Oncology, pp. 221-229 (March, 1978).

Cellulite is described by various stages or degrees as follows. Degree 0 means no alterations appear on the skin surface, even when the pinch test or muscular contraction is employed. Degree I means no alterations appear on the skin surface when untouched and not contracted, but the orange-peel phenomenon appears on the skin from the pinch test or from muscular contraction. Degree II means the orange-peel phenomenon is visible to the naked eye, without the pinch test and without muscular contraction on the skin surface. Degree III means the presence of the same alterations as described in Degree II, but also with raised areas and nodules. See, Hexsel et al., “Subcision: A Treatment for Cellulite”, Vol. 39, International Journal of Dermatology, pp. 539-544 (2000).

The cause of cellulite is due to the composition of skin structure. As adipose tissue (i.e., fat tissue) increases, the adipose tissue expands into the upper layers of the skin, namely the dermis. See, Rosenbaum et al., supra. The expansion of adipose tissue results in protrusion of the overlying dermis. See, Numberger et al., supra. Along with the adipose tissue, typically a slight edema in the cellulite-affected area occurs. The edema is caused by the retention of water in the skin. Also, sometimes, frameworks of collagen form, resulting in subsequent sclerosis. See, Lotti et al., “Proteoglycans in So-Called Cellulite”, Vol. 29, No. 4, International Journal of Dermatology, pp. 272-274 (May, 1990).

In short, cellulite is not some mysterious skin condition. Despite popular advertisements which assert that cellulite is alleviated by releasing trapped toxins or by improving poor blood circulation, cellulite actually results, according to current accepted medical theory, from just two simple things—fat and retained water. Cellulite-affected skin looks different from skin not so affected merely due to how the adipose tissue is arranged. Everyone has strands (i.e., fibers) of connective adipose tissue that separate fat cells in the skin. In women, the fibers form a honeycomb pattern, so that any increase of the adipose tissue in a given area tends to bulge. In general, men do not exhibit cellulite because their fibers run in a horizontal, crisscross pattern that prevents bulging or dimpling.

Cellulite seems to appear out of nowhere and to become worse as a person becomes older because (1) the connective fibers of tissue thicken, (2) the skin becomes thinner, and (3) muscle is lost and replaced with fat. The last is particularly a problem for women, who tend to lose 5 pounds of muscle and replace it with 15 pounds of fat every decade of adult life.

Currently, there are, in general, three methods for the treatment of cellulite-affected skin, namely surgery, creams (or gels) topically applied, and massage (or endomologie).

Surgery generally falls into two types, which are subcision surgery and liposuction surgery.

With subcision, needles are employed to make incisions into the deep layer of subcutaneous fat and remove it. A local anesthetic is used. Bruising usually occurs and typically lasts from 3 to 6 months. A compression belt must be worn on the treated area for 2 to 3 weeks after surgery to help reduce the bruising. Sometimes, an adverse side effect is elevation of the treated areas. See, Hexsel et al., supra.

With liposuction, incisions are made, followed by inserting a cannula into each incision to break up the fat, and then to suction it out. A local anesthetic is used and bruising may occur. Firm bandages are used to reduce the risk of postoperative bleeding and fluid accumulation. Results, however, may take up to several months to become visible after liposuction. Sometimes, an adverse side effect is that the treated skin becomes even bumpier. See, Lewis, Collier, and Heitkemper, Medical Surgical Nursing: Assessment and Management of Clinical Problems, Fourth Edition, Mosby Publishing, St. Louis, Missouri, pp. 515 and 1131 (1996).

A cream and/or gel, such as aminophylline-containing cream, which is advertised as a cosmetic that removes cellulite from the thighs, can be used as a topical treatment. See, Dickinson and Gora-Harper, “Aminophylline for Cellulite Removal”, Vol. 30, The Annals of Pharmacotherapy, pp. 292-293 (March, 1996). A drawback is that topical treatment with creams and/or gels leaves a greasy-feeling residue that messes clothing.

Endomologie is a deep massage that appears to help temporarily with smoothing out the clumping of fat by high technology rolling and suction. However, a drawback is the time involved in repeat treatments, as well as the cost, typically about $1600 for 20 treatments of 40 minutes each.

Hence, it is still desirable to find a method of treating cellulite-affected skin topically that does not require abrasion and debridement of the affected skin (which can result in redness and swelling from irritation of the affected skin) and that does not require oral ingestion of a medicament (which can result in undesirable side effects, such as nausea, dizziness, and hypertension) and that does not require surgery (which has various risks, such as bruising, infection, and bleeding, as well as sickness or death from anesthetic).

### SUMMARY AND OBJECTS OF THE INVENTION

Accordingly, the present invention provides a method for the topical treatment of skin affected with cellulite. The method comprises selecting a solid block formulation of sodium chloride, potassium chloride, or a combination thereof, and topically applying the solid block to the affected skin by gently gliding the solid block over the affected skin, in a manner insufficient to cause abrasion and debridement of the affected skin. The applied salt selected from the group consisting of sodium chloride, potassium chloride, and a combination thereof is allowed to remain on the affected skin. An improvement in the cellulite is achieved by the method, and often the cellulite is eliminated. Preferably, the application of the formulation is for about 50 seconds or less. Also preferably, the formulation comprises a substantially pure form of NaCl and should contain about 80% to about 100% NaCl, more preferably about 95% to about 100% by weight of NaCl as a solid block. Nevertheless, amounts of NaCl that are less than 80% by weight NaCl may desirably be present when potassium ion is also present as discussed below. Furthermore, the formulation should be free of a carrier and/or free of other topical treatments for cellulite. Specifically, granular NaCl, KCl, or a combination thereof should not be employed, as that may cause debridement.

The following should be done in order to assist in a portion of the NaCl and/or KCl leaving the block and forming a film or coating on the cellulite-affected skin. More specifically, prior to gliding the block over the affected skin, the skin is preferably pre-moistened with water, such as from shaving, bathing, splashing water on the affected skin, and the like. Then, the coating of NaCl and/or KCl applied to the affected skin is allowed to dry on the affected skin.

In one embodiment, the formulation of NaCl and/or KCl includes one or more other ingredients that are the kind of ingredients naturally present in human extracellular fluid and/or human intracellular fluid, as discussed below.

In still another embodiment, the formulation of NaCl and/or KCl includes another ingredient such as a salt of S, P, Zn, Mn, Fe, Cu, Cr, I, Co, Mg, Ca, and/or Se.

Therefore, it is an object of the present invention to treat cellulite-affected skin topically with NaCl and/or KCl without any concomitant debridement that can easily result in red, irritated skin.

It is a further object of the present invention that NaCl and/or KCl used in the skin treatment is not orally ingested, and hence the present invention obviates the risk of nausea, dizziness, or hypertension that can result from oral ingestion of too much NaCl and/or KCl.

It is a feature of the present invention that the NaCl and/or KCl treatment obviates the deep massage or endomologie that, even though quite expensive, is frequently used to treat cellulite-affected skin by smoothing out the clumping of fat that causes cellulite.

Moreover, it is an important advantage of the present invention that persons who have cellulite can avoid surgery, and the attendant risks of surgery, such as bruising or infection or such as the anesthetic causing adverse effects that can be as severe as death.

Some of the objects, features, and advantages of the invention having been stated above, others will become evident as the description proceeds, when taken in conjunction with the Laboratory Examples as best described below.

### DETAILED DESCRIPTION OF THE INVENTION

As the present invention involves topical treatment, thus surgery, deep massage, and the like, commonly used in treating cellulite, advantageously are avoided, as well as oral ingestion of drugs is avoided.

cellulite-affected skin area, such as the thighs, buttocks, sides, or arms of a human, may be topically treated with the method of the present invention. The method may be employed for any variation of cellulite, whether mild or severe or somewhere in between. Furthermore, the method works similarly well in the treatment of both males and females who have cellulite.

More specifically, the inventive method involves topically applying a solid block formulation comprising a salt selected from the group consisting of NaCl, KCl, and a combination thereof to the affected skin.

Preferably, a solid block of substantially pure NaCl, KCl, or a combination thereof should be used. In other words, the block should contain between about 80% and about 100%, more preferably between about 95% and about 100%, even more preferably between about 98% and about 100%, by weight NaCl, KCl, or a combination thereof, optionally, with only trace amounts present of other ingredients such as sulfur or mineral salts, for instance salts of Mg or Ca.

In particular, potassium ion may be present in a Na:K ratio by weight of up to about 1:1. For instance, a solid block may contain about 57.5% by weight NaCl and about 42.5% by weight KCl, or a solid block may contain about 46% by weight NaCl, about 34% by KCl, and about 20% by weight of one or more of various other ingredients such as a salt of S, P, Zn, Mn, Fe, Cu, Cr, I, Co, Mg, Ca, and/or Se. It is noted that these percents of NaCl and KCl result in a Na:K ratio of about 1:1. Each of the various other ingredients, such as S, P, Zn, Mn, Fe, Cu, Cr, I, Co, Mg, Ca, and/or Se, may be individually present in a trace amount up to about 1.8%-1.9%, which would add up to about 20% by weight if all were present.

However, the solid formulation of NaCl and/or KCl may contain between about 55% and about 100% by weight, preferably about 65% and about 100% by weight, more preferably about 75% to about 100% by weight, and even more preferably about 95% to about 100% by weight NaCl and/or KCl, together with containing one or more of various other ingredients that are the kinds of ingredients naturally present in human extracellular fluid (plasma fluid or interstitial fluid) and/or in human intracellular fluid. Such ingredients from human body fluid include, but are not limited to, potassium salts (other than the KCl salt), sodium salts (other than the NaCl salt), other sodium complexes, and/or other potassium complexes. As discussed in Review of Medical Physiology, Ganong, 18^{th} Edition, Chapter 1 (The General and Cellular Basis of Medical Physiology), pp. 27-28 (1997), human body fluid naturally contains, in addition to NaCl and KCl, ingredients such as potassium carbonate, potassium protein complexes, potassium phosphate, sodium carbonate, sodium protein complexes, and sodium phosphate.

The amount of such human body fluid ingredients other than NaCl and/or KCl should be less than about 45%, more preferably less than about 35%, and even more preferably less than about 25% by weight, based on the weight of the NaCl and/or KCl. Thus, the amount of NaCl and/or KCl would be more than about 55%, more preferably more than about 65%, and even more preferably more than about 75% by weight.

A discussion of a solid formulation of NaCl, together with potassium and/or various other ingredients that are naturally present in human extracellular fluid and human intracellular fluid can be seen in applicant's co-pending U.S. patent application Ser. No. 09/721,131, filed Nov. 22, 2000, to Bass, the disclosure of which is incorporated by reference. Of course, both kinds of the various other ingredients may be present, namely one or more of the other salt ingredients such as a salt of S, P, Zn, Mn, Fe, Cu, Cr, I, Co, Mg, Ca, and/or Se, and one or more of the other ingredients naturally contained in human body fluid such as potassium carbonate, potassium protein complexes, potassium phosphate, sodium carbonate, sodium protein complexes, and sodium phosphate.

For use in the method of the present invention, the block of NaCl and/or KCl also should be free of any carriers. Typical pharmaceutically acceptable carriers (such as ethanol, glycerol, stearyl alcohol, and glycerylmonostearate, often used to place a cosmetic or medicament in solution form or emulsion form for application) need not be used for the present invention, and preferably, are not used.

Even more preferably, the block of NaCl and/or KCl also should be free of any other medicaments or cosmetics for the topical treatment of cellulite.

Additionally, the block of NaCl and/or KCl should be of appropriate size to be conveniently held in the hand of the user so that the block may be gently glided onto the affected skin. A convenient size may range from a cubic shape of about 0.5 inch on a side to a rectangular parallelapiped shape of about 1 inch×1 inch×2 inches, and of course, other convenient shapes, such as cylinders, may be used.

Substantially pure solid blocks of KCl useful in the method of the present invention may be like the 1500 mg or 750 mg KCl tablets sold by Key Pharmaceuticals, Incorporated of Kenilworth, N.J., under the registered trademark K-DUR, for oral administration to persons who need extra potassium in their diet, for instance persons who are taking diuretics. However, the solid blocks for use in the present invention would be larger, i.e., a convenient size to be held in the hand of the user, as described in the above paragraph.

Substantially pure NaCl naturally occurs as rock salt, also known as the mineral, halite. Halite is isometric with cubic habit and cleavage. It is translucent when pure, but may be white, yellow, red, or blue when trace amounts of other minerals are present.

Solid blocks of NaCl useful in the method of the present invention are described in U.S. Pat. Nos. 5,869,104 (issued Feb. 9, 1999) and 5,654,013 (issued Aug. 5, 1997), both to Taylor and Bass, the disclosures of both of which are incorporated by reference.

In particular, a block of NaCl, which is useful in the method of the present invention, is described in detail in U.S. Pat. No. 5,869,104. More specifically, this patent discusses that Morton Salt of Chicago, Ill. makes and markets substantially pure NaCl blocks under the registered trademark BUNNY SALT SPOOLS. Certain BUNNY SALT SPOOLS are plain, white, 3-ounce spools having a generally cylindrical shape (i.e., a beveled disc with a center pinhole), which are manufactured by compressing vacuum granulated salt. The white spools have a minimum purity of 99.0% NaCl, and may contain certain small amounts of iodide and/or salts of S, Zn, Mn, Fe, Cu, and/or Co. These white spools are particularly useful in the method of the present invention.

Morton Salt markets the white spools (as well as pink spools under the registered trademark IOFIXT and red spools under the registered trademark IOFIXT T-M) for use in feeding NaCl to small animals such as rabbits, mink, guinea pigs, and chinchillas. The pink and/or red spools could be used in the present invention, but the color is probably due to the presence of food coloring, and thus, such colored spools probably would leave undesirable color spots on the skin of the person.

Additionally for feeding NaCl to small animals, Morton Salt makes and sells yellow spools, which are yellow due to the presence of about 3% sulfur salt compounds and which are about 95% to 97% NaCl. The yellow spools should also be useful in the method of the present invention.

The NaCl, KCl, or combination thereof, preferably in the form of a block, is topically applied by very gentle gliding motions, such as dabbing motions, circular motions, up and down motions, or zigzag motions, as the NaCl and/or KCl is gently glided over the affected skin. Care must be taken so that gently gliding the NaCl and/or KCl over the affected skin is in a manner insufficient to cause abrasion and debridement of the affected skin.

The gliding should be accomplished in about 50 seconds to about 1 second, and more preferably in about 45 seconds to about 2 seconds. Typically, the gliding will be accomplished in about 30 seconds to about 15 seconds.

After the block of NaCl and/or KCl has been glided over the affected skin, the applied NaCl and/or KCl is left on the skin. Typically, the applied NaCl and/or KCl forms a thin film or coating of NaCl and/or KCl over the affected skin

Prior to gliding the block of NaCl and/or KCl on the affected skin, the skin should be pre-moistened with water to assist a portion of the NaCl and/or KCl in leaving the block and staying applied on the affected skin. Then, the applied NaCl and/or KCl will have been wetted, and is left to dry on the skin. The pre-moistening can be accomplished, such as by a female shaving the legs and then after splashing water on the legs to remove residual shaving cream, not drying the legs. Additionally, the affected skin can be pre-moistened by simply leaving it wet after taking a bath or a shower. Furthermore, the affected skin can be pre-moistened by simply splashing water on the affected skin.

Application to the affected skin should be at least once during a day, but may be oftener depending on the severity of the cellulite. Hence, application may be as often as 5 or 6 times during a day, or even more. Typically, for most persons affected with cellulite, application once or twice during a day is sufficient.

The applied NaCl and/or KCl should not be removed, but rather left on the skin until the next time the person takes a shower or bath, which is usually once per day.

The application should be repeated for at least 1 week, preferably at least 2 weeks, on a regular basis, and the cellulite will have been alleviated and often eliminated. For a severe case of cellulite, application on a regular basis should be for at least 2 times during a day and for at least 5 weeks to eliminate the skin condition of cellulite.

After elimination of the cellulite, application may be once during a day to maintain the skin cellulite-free.

### LABORATORY EXAMPLES

### Example I

A female person, Caucasian, 45 years old, who had cellulite-affected skin on her thighs and buttocks, was treated by the inventive method. A solid block of mineral pure rock salt that contained at least 95% NaCl was used. It was white and was obtained from Morton Salt. The testing of the subject was as follows.

Once per day, after she finished taking her shower, and was done with washing with soap and rinsing with water, she did not dry her thighs and buttocks. Rather, these areas were left moistened. Then, she applied NaCl from the block to the cellulite-affected areas of the thighs and buttocks by very gently gliding the block of NaCl in dabbing motions, circular motions, up and down motions, or zigzag motions (the motions being insufficient to cause debridement and abrasion of the affected skin) on the moistened areas for about 15 to about 30 seconds. A thin film of wetted NaCl resulted, and was allowed to remain and to dry on affected skin. This once daily application of NaCl was continued for 5 weeks. At the end of 2 weeks, the cellulite was alleviated with the skin beginning to appear smoother, and at the end of 5 weeks, the NaCl treated areas were free of cellulite, which had been completely eliminated so that the skin looked very smooth.

She continued with the once per day application of NaCl and her thighs and buttocks remained free of cellulite.

### Example II

The method may be repeated with more females in the same manner of application as with the female test subject noted above in Example I, either once per day, twice per day, or more times, depending on the severity of the cellulite, but instead with female persons not having the cellulite-affected areas of the thighs and buttocks pre-moistened with water, but instead dry, and the results should be the same.

### Example III

The method may be repeated with more females in the same manner of application as with the female test subject noted above in Example I, either once per day, twice per day, or more times, depending on the severity of the cellulite, but instead with female persons using a solid block of KCl instead of NaCl, and the results should be the same.

### Example IV

The method may be repeated with more females using KCl in the same manner of application as with the female test subjects noted above in Example m, either once per day, twice per day, or more times, depending on the severity of the cellulite, but instead with female persons not having the cellulite-affected areas of the thighs and buttocks pre-moistened with water, but instead dry, and the results should be the same.

### Example V

This should happen when a male test subject is treated with a block of NaCl, like the block employed in Example I above. This person may be a 50-year old male Caucasian afflicted with cellulite on his sides, between his armpits and his waist.

Once per day, after he washes in the shower with soap and water, and then rinses with water, he does not dry his sides. Rather, his sides are left moistened. Then, he applied NaCl from the block to the cellulite-affected areas of his sides, by very gently gliding the block in dabbing motions, circular motions, up and down motions, or zigzag motions (the motions being insufficient to cause debridement and abrasion of the affected skin) on the moistened areas for about 15 to about 30 seconds. A thin film of wetted NaCl will result, and is allowed to remain and to dry on his sides.

This once daily application is continued for 5 weeks. At the end of 1 or 2 weeks, the cellulite should begin to be alleviated. At the end of 5 weeks, his sides should have the cellulite completely eliminated. He should continue with the once per day application of NaCl and his sides should remain smooth and free of cellulite.

### Example VI

The method may be repeated with another male in the same manner of application as with the male test subject noted above in Example V, either once per day, twice per day, or more times, depending on the severity of the cellulite, but instead with a male person using a solid block of KCl instead of NaCl, and the results should be the same.

It will be understood that various details of the invention may be changed without further departing from the scope of the invention. Furthermore, the foregoing description is for the purpose of illustration only, and not for the purpose of limitation—the invention being defined by the claims.

What is claimed is:

1. A method for the topical treatment of skin affected with cellulite comprising the steps of:
(A) selecting a solid block formulation comprising a salt selected from the group consisting of sodium chloride, potassium chloride, and a combination thereof,
(B) applying the solid block by gently gliding it over the affected skin in a manner insufficient to cause abrasion and debridement of the affected skin,
(C) allowing the applied salt selected from the group consisting of sodium chloride, potassium chloride, and a combination thereof to remain on the affected skin, and
(D) achieving an improvement in the cellulite.

2. The method of claim 1, wherein the block contains about 95% to about 100% of a salt selected from the group consisting of sodium chloride, potassium chloride, and a combination thereof

3. The method of claim 1, wherein the block of a salt selected from the group consisting of sodium chloride, potassium chloride, and a combination thereof is free of other mineral salts except for trace amounts of the other mineral salts.

4. The method of claim 1, wherein the block of a salt selected from the group consisting of sodium chloride, potassium chloride, and a combination thereof comprises another ingredient selected from the group consisting of a salt of Mg, Ca, S, P, Zn, Mn, Fe, Cr, I, Cu, Co, Se, and combinations thereof, in a total amount up to 20% by weight.

5. The method of claim 1, wherein the block of a salt selected from the group consisting of sodium chloride, potassium chloride, and a combination thereof is free of a carrier.

6. The method of claim 1, wherein the block of a salt selected from the group consisting of sodium chloride, potassium chloride, and a combination thereof is free of other topical medicaments and cosmetics for the topical treatment of cellulite.

7. The method of claim 1, wherein gliding the block of a salt selected from the group consisting of sodium chloride, potassium chloride, and a combination thereof over the affected skin is accomplished by gentle rubbing motions selected from the group consisting of dabbing motions, circular motions, up and down motions, zigzag motions, and combinations thereof

8. The method of claim 1, wherein gliding the block of a salt selected from the group consisting of sodium chloride, potassium chloride, and a combination thereof over the affected skin is accomplished in about 50 seconds to about 1 second.

9. The method of claim 8, wherein gliding the block of a salt selected from the group consisting of sodium chloride, potassium chloride, and a combination thereof over the affected skin is accomplished in about 45 seconds to about 2 seconds.

10. The method of claim 1, wherein the applied salt from the block of a salt selected from the group consisting of sodium chloride, potassium chloride, and a combination thereof forms a film on the affected skin.

11. The method of claim 1, wherein prior to gliding the block of a salt selected from the group consisting of sodium chloride, potassium chloride, and a combination thereof over the affected skin in step (B), the affected skin is pre-moistened with water, and then, gliding the block of sodium chloride, potassium chloride, and a combination thereof over the affected skin results in the sodium chloride, potassium chloride, and combination thereof becoming wet, and then in step (C), the wetted applied sodium chloride, potassium chloride, and combination thereof remaining on the affected skin is allowed to dry on the affected skin.

12. The method of claim 1, wherein steps (A), (B), and (C) are accomplished at least once per day.

13. The method of claim 1, wherein the block of a salt selected from the group consisting of sodium chloride, potassium chloride, and a combination thereof comprises sodium chloride and potassium chloride in a weight ratio amount of Na:K up to about 1:1.

14. The method of claim 1, wherein the block of a salt selected from the group consisting of sodium chloride, potassium chloride, and a combination thereof comprises another ingredient selected from the group consisting of an ingredient naturally present in human extracellular fluid, an ingredient naturally present in human intracellular fluid, and a combination thereof

15. The method of claim 14, wherein the other ingredient naturally present in human fluid is selected from the group consisting of potassium carbonate, potassium protein complexes, potassium phosphate, sodium carbonate, sodium protein complexes, sodium phosphate, and combinations thereof, wherein the total amount of said other ingredients is less than about 45% by weight, based on the weight of the block of sodium chloride, potassium chloride, and a combination thereof
